(19) United States
(12) Patent Application Publication
(10) Pub. No.: US 2006/0088594 A1
(43) Pub. Date: Apr. 27, 2006

(54) Highly compressible controlled delivery compositions of metformin

(76) Inventors: Pratibha Sudhir Pilgaonkar, Mumbai (IN); Maharukh Tehmasp Rustomjee, Mumbai (IN); Anilkumar Surendrakumar Gandhi, Mumbai (IN); Atul Anand Kelkar, Mumbai (IN); Vinderjit Sarjit Bhumra, Mumbai (IN)

Correspondence Address: John C. Serio;Brown Rudnick Berlack Israels LLP, One Financial Center, Box IP, Boston, MA, 02111, US

(21) Appl. No.: 11/246,866

(22) Filed: Oct. 7, 2005

Related U.S. Application Data
(60) Provisional application No. 60/617,241, filed on Oct. 8, 2004.

(51) Int. Cl. A61K 9/20

(52) U.S. Cl. 424/464

(57) ABSTRACT

Highly compressible controlled delivery compositions of metformin or salts thereof and the process of making the same are disclosed. Metformin is granulated with a binder and further dispersed in a rate-controlling matrix that results in increased hardness and decreased friability thereby effectively solving compressibility difficulties arising for Metformin formulations.

### CROSS-REFERENCE TO RELATED APPLICATIONS

This application claims priority to U.S. Provisional Patent Application Ser. No. 60/617,241, filed Oct. 8, 2004 the entire contents of which are incorporated herein by reference.

### FIELD OF INVENTION

The present invention relates to highly compressible controlled delivery compositions of pharmaceutical therapeutics and the process of making the same. In particular, the present invention relates to metformin and salts thereof essentially granulated with a binder and further dispersed in a rate-controlling matrix that results in increased hardness and decreased friability thereby effectively solving compressibility difficulties arising for metformin formulations.

### BACKGROUND OF INVENTION

Metformin Hydrochloride (N,N-dimethylimidodicarbonimidic diamide hydrochloride) is a white to off-white crystalline compound with a molecular formula of C₄H₁₁N₅.HCl and a molecular weight of 165.63. It is freely soluble in water and is practically insoluble in acetone, ether, and chloroform. Metformin is a biguanide that is not chemically or pharmacologically related to any other classes of oral anti-hyperglycemic agents. It is absorbed mainly from the small intestine.

Metformin is stable in vivo and it does not bind to plasma proteins and is therefore excreted unchanged in the urine. It has a half-life of 1.3 to 4.5 hours. The maximum recommended daily dose of metformin is 3 gms. Metformin is anti-hypererglycemic and it improves glucose tolerance in patients with type II diabetes, lowering both basal and postprandial plasma glucose. Unlike sulfonylureas, metformin does not produce hypoglycemia in either patients with type II diabetes or normal subjects. Hence it is a drug of choice in controlling type II diabetes and is widely prescribed by physicians all over the world.

As with substantially all pharmaceutical therapeutics, patient compliance is an primary concern with controlling diabetes with oral anti diabetics. As is well known in the art, the need to administer therapeutics in multiple doses contributes to a high rate of non-compliance. The convenience of administering a single dose of a medication, which releases active ingredients in a controlled fashion over an extended period of time, as opposed to the administration of a number of single doses at regular intervals, has long been recognized in the pharmaceutical arts as improving patient compliance.

The advantage to the patient and clinicians in having consistent and uniform blood levels of medication over an extended period of time are likewise recognized. Among the most important advantages are: increased contact time for the drug to allow for local activity in the stomach, intestine or other locus of activity; increased and more efficient absorption for drugs which have specific absorption sites; the ability to reduce the number of dosages per period of time; employment of less total drug; minimization or elimination of local and/or systemic side effects; minimization of drug accumulation associated with chronic dosing; improved efficiency and safety of treatment; reduced fluctuation of drug level; and better patient compliance with overall disease management.

Management of hyperglycemia requires regular dosing over the patient's life-time and it is therefore essential to have a controlled release formulation of anti-hyperglycemic agents like metformin that reduces the frequency of dosing thereby improving patient compliance. Unfortunately, metformin is a compound having high aqueous solubility, this high aqueous solubility coupled with the need for relatively a high dose and poor compressibility poses considerable challenges in formulating a controlled release system. Since solubility is the primary factor for drugs to dissolve in water therefore greater solubility results in a greater rate of dissolution.

Controlled release systems are generally monolithic systems where the drug is embedded in polymeric matrix. Unfortunately, in order to control the rate of release of drug having high water solubility, very large amounts of polymer are required for the matrix. While this may be feasible for highly soluble low dose drugs, high dose drugs with high solubility require substantially higher quantities of polymers in the matrix. The need of high quantities of polymers within a matrix, leads to unacceptable larger dosage forms. These large dosage forms lead to patient compliance problems and safety problems associated with such a large dosage form.

Another problem in the formulation of extended release metformin is that it is an inherently hygroscopic compound that has compressibility-related problems. Excessive fines in the compression blend and high compression pressures further add to the problems leading to capping, low hardness tablets that have low friability. Poor compressibility thus adds to the manufacturing costs as the tableting process is not rugged and therefore requires constant monitoring to avoid breakdowns or maintain compositions within the pre-specified quality control limits.

Attempts to formulate metformin into a liquid dosage form have proven to be ineffective for controlled release formulations. Also metformin blended into a powder form and filled into capsules is has also proven to be not useful in a controlled or extended release formulation, since an effective gastro-retentive system cannot be formed from loosely packed powder.

While that has been some success within the prior art of controlling the release of highly soluble drug like metformin, none of the prior art methods thus far have addressed the problems associated with compressibility successfully.

U.S. Pat. No. 6,475,521 to Timmins et al. (Timmins), describes a method of preparing a bi-phasic controlled release metformin tablet. The method of Timmins comprises forming a discrete inner solid particulate phase in form of individual particles containing metformin and an extended release material and mixing these individual particles with an outer solid continuous phase comprising an extended release material in which the particles of the inner solid particulate phase are dispersed and embedded.

The discrete two-phase inner and outer system of Timmins is believed to address the initial “burst” of a highly soluble drug that can occur from a controlled release system. The burst of highly water soluble drug is the initial rapid release of drug that occurs from oral controlled release dosage forms when first contacting fluid, prior to the release controlling mechanisms of the dosage form establishing themselves thereby providing a stable release rate.

In the discrete bi-phasic system of Timmins, the drug released from the particles of the inner phase is believed to migrate through the outer solid continuous phase and is then released allowing a continuous controlled release. As explained by the specification of Timmins, it is therefore a requirement that the rate-controlling polymer in the inner solid continuous phase is in substantial quantity to control the initial burst and to maintain the controlled release.

U.S. Pat. No. 5,955,106 to Moeckel et al. (Moeckel) describes an extended release tablets of metformin hydrochloride that are prepared by a wet granulation process. The Moeckel process comprises granulating metformin and a hydrocolloid forming retarding agent with an aqueous solvent to form a granulated product and then drying the granulated product to residual moisture content of about 0.5 to 3% by weight. The Moeckel process is believed to alleviate . . . 0.capping of tablets during manufacturing by critically controlling the moisture content prior to tableting. Unfortunately, the need to contain the moisture content within a critical range contributes to other manufacturing problems.

US Patent Application No. 2003/0104059 A1 describes controlled release tablets of metformin and a process of forming the same. The process comprises of dry blending metformin with hydrophilic polymers consisting of anionic and nonionic polymers in a ratio 1:1 to 1:5, and optionally other excipients, granulating the blend, drying and sizing the granules and compressing to make tablets, wherein at least about 16% by weight of the composition is the hydrophilic polymer.

WO 03/099214 A2 describes pharmaceutical dosage form consisting essentially of a single-phase matrix comprising metformin or a pharmaceutically acceptable salt thereof and at least one controlled release excipient. Examples demonstrate that all the ingredients are mixed together and tablets are made either by direct compression or by wet granulation using aqueous or organic solvents.

US20040202718 describes a dosage form comprising a compressible controlled release core composition comprising metformin, two or more swellable polymers wherein at least one polymer is an anionic polymer, one or more pharmaceutically acceptable excipient(s) that improve the compressibility of the core composition and optionally a coat comprising one or more water insoluble polymer(s) surrounding the core. The desired retardation in release is not achieved by matrix formulation alone and therefore a water insoluble coating is desired which increases manufacturing process steps and costs. Also the manufacturing process involves wet granulation with polymers or direct compression, which have disadvantages as mentioned with other prior arts.

WO05060942 relates to a process for the preparation of a matrix of polymer and carbonate along with metformin and optionally with fillers, disintegrants in rapid mixer granulator and granulating the resulting blend with solution of binder in aqueous or non aqueous solvent, drying the granules, lubricating the dried granules with lubricants compressing the lubricated blend to form extended release tablets. The formulation describes a floating delivery system in which the problem of compressibility of metformin is not addressed and metformin is granulated with polymers.

The above prior art describes controlled release formulations made using wet granulation techniques with the rate controlling polymers completely or partially granulated with the drug. Unfortunately, granulation of the drug and the rate-controlling polymeric mass with water results in surface gelling of the wetted polymers leading to formation of agglomerates. These agglomerates are difficult to pass through the sieve and after drying they tend to leave elastic mass on the screen. Formation of elastic mass results in reduced efficiency of these polymers and larger amounts of polymers are required for retarding the drug release. One approach of reducing the elastic mass formation is to use organic solvents, however use of organic solvents is not considered viable due to complexities involved in removal of traces of the solvent from the product as well as environmental hazards.

It has been found that these above compositions do not sufficiently improve the compression characteristics of drug such as metformin and the resultant tablets are likely to face problems in manufacturing and have friability issues. As metformin is bitter in nature, poor friability is an important concern if the tablets are to be coated; these tablets may break during coating.

Additionally granulation of drug with polymers may pose problems in equipment cleaning and operation since the polymeric mass tends to be sticky. This problem is particularly serious with above wet granulation prior art processes as rate retarding polymers are employed for the granulation of metformin at higher concentration for them to act as release retardants for highly soluble metformin hydrochloride. These rate-controlling polymers are usually high molecular weight polymers, which forms elastic/gummy mass upon hydration with water. Hence special precautions or cleaning procedures are likely to be employed for cleaning the equipments after granulation.

Further attempts to formulate metformin have been directed towards direct compression. U.S. Pat. No. 6,524,618 describes use of specific excipients of particular size and density range to improve the flow and compressibility of metformin hydrochloride. These excipients are blended with metformin and then the blend is then directly compressed. The use of excipients with a specific particle size and density range makes the manufacturing process tedious and expensive. Additionally metformin needs to be of a particular particle size i.e. 150-600 microns, which is an additional manufacturing step which makes the process cost prohibited. Additionally, this directed compression method may not work for metformin having particle size outside this range.

Slugging has also been tried in prior art methods to balance the compressibility and high solubility issues of metformin. US Patent Application No. 2004/0059001A1 describes an extended release compositions of metformin comprising metformin and a rate-controlling polymer. This process comprises the moisture conditioning of metformin, wherein the pharmaceutical composition has water content of from 3.2% to about 10% by weight, alone or its blend with rate controlling polymer and pharmaceutically acceptable excipients and further subjecting the blend to compaction or slugging.

Unfortunately, Slugging or compaction adds additional process steps and therefore manufacturing costs. Furthermore, the process does not give satisfactory increase in compressibility to the poorly compressible metformin and hence has not proven to be beneficial.

Other prior art methods have used a wax matrix for formulating a controlled release dosage form for addressing the compressibility problems. US Patent Application No. 2004/0086566 describes wax matrix dosage forms, the matrix comprising metformin or a pharmaceutically acceptable salt thereof and a wax matrix material. The wax material is preferably prepared by hot melting a suitable wax material and using the melt to granulate the metformin material.

Another method uses waxes in the formulation is described in WO 03/004009 A1 which discloses a process for preparing a pharmaceutical tablet of a poorly compressible pharmaceutical agent like metformin formulated as a monolithic or single phase system comprising preparing a blend of metformin, a hydrophilic erodable component and a hydrophobic component and compressing the blend into tablet. Any process other than wet granulation can be used and the examples show melting stearyl alcohol wax and granulating the drug with the melted wax. However wax matrix formation is an energy intensive process as high temperatures are required to melt waxes. Moreover waxes inherently have poor compressibility owing to their plastic/elastic nature. Combination of poor compressible metformin and wax is likely to further reduce the compressibility of the final blend.

The above prior art approaches reveal that various attempts have been made to make controlled release formulations of metformin or a pharmaceutically acceptable salt thereof using varying techniques such as: wet granulation, wherein the rate controlling polymers are completely or partially granulated with the drug; direct compression, using specific excipients of particular size and density range or as such; dry granulation, as such or involving slugging or compaction with moisture conditioning of metformin; and wax matrix formation. Unfortunately all of these compositions and methods utilizing various polymers and matrices are cost-intensive and cumbersome techniques of manufacturing that have not been shown to be successful in imparting good compression characteristics to the metformin blend.

### SUMMARY OF THE INVENTION

The present invention addresses the compressibility problems with metformin and provides a simple compositions of metformin which are inexpensive to process and yet have excellent compressibility characteristics thereby giving tablets with negligible friability and good hardness at lower compression pressures that have comparable bioavailability profiles to the marketed formulation.

It has been surprisingly found that essentially granulating metformin alone with about 0.1% to about 10% binder, preferably about 0.2% to about 5% binder and most preferably about 0.25% to about 4.5% binder prior to addition of the rate-controlling polymers substantially increases compressibility as compared to prior art formulations where metformin is compressed after either directly granulating or directly mixing with all the extended release polymers. The issues related to compressibility and controlling release due to high solubility are successfully tackled by the simple to manufacture and cost effective compositions of the present invention. According to an aspect of the present invention there is provided a highly compressible controlled release composition of metformin or a pharmaceutically acceptable salt thereof comprising approximately 98 percent of the total metformin within the formulation granulated with about 0.1% to about 10% binder, preferably about 0.2% to about 5% binder and most preferably about 0.25% to about 4.5% binder and then mixed with a rate-controlling matrix of hydrophilic polymers and approximately 2 percent of the total metformin within the formulation in a free unassociated form (free metformin). According to the present invention, the metformin granules are essentially bound with the binder, and further dispersed in the rate-controlling matrix of hydrophilic polymers and the free metformin, wherein the hardness achieved by compressing these compositions into tablets is at least 8 kg/cm2.

According to an aspect of the present invention, there is provided a process for preparing the said composition according to the invention comprising essentially granulating metformin with about 0.1% to about 10% binder, preferably about 0.2% to about 5% binder and most preferably about 0.25% to about 4.5% binder using water, drying and sizing bound metformin granules and dispersing the granules in a blend essentially comprising one or more rate-controlling hydrophilic polymers along with approximately 2 percent free metformin.

According to a third aspect of the present invention, there is provided a method of treatment of hyperglycemic patients comprising administering the tablets of varied dose made according to the invention once daily to patients in need thereof.

It is thus an object of the present invention to provide controlled release compositions of metformin or a pharmaceutically acceptable salt thereof that have excellent compressibility characteristics resulting in tablets having high hardness and negligible friability.

Another object of the present invention is to provide controlled release compositions of metformin that effectively control the release of highly soluble metformin yet provide good compressibility without the use of cumbersome methods of prior art.

A further object of the present invention is to provide controlled release compositions of metformin that are simple to manufacture without involving cost intensive methods of preparation yet giving superior compressibility, hardness and reduced friability over prior art formulations prepared using cost effective methods.

Yet another object of the present invention is to provide a process for preparing a controlled release compositions of metformin where the metformin is essentially granulated with a suitable binder prior to incorporating in controlled release system.

Another object of the present invention is to provide a process for preparing the said controlled release compositions of metformin where the process is independent of the particle size of the metformin.

A further object of the present invention is to provide controlled release compositions of metformin that have comparable bioavailability profiles with existing marketed formulation.

An additional object of the present invention is to provide controlled release compositions of metformin that can utilize the rate controlling polymers in a manner resulting in a reduced tablet weight.

Yet another object of the present invention is to provide controlled release compositions of metformin that can give dose weight proportionate formulations of varying strength.

A further object of the present invention is to provide controlled release compositions of metformin that have negligible friability.

Another object of the present invention is to provide controlled release compositions of metformin that are simple to manufacture and result in good hardness tablets that can withstand the rigors of coating.

The present invention is a simple solution to the compressibility related to metformin formulations which is cost effective, simple to manufacture, does not utilize costly or heat intensive techniques and yet gives excellent formulation characteristics in the form of improved compressibility and tablet hardness with negligible friability. It further surprisingly demonstrated that both the major issues related to metformin of compressibility and formulating in a controlled release manner due to high solubility, are easily solved by the present compositions without the need of metformin granulation separately with extended release polymers to control initial burst, disadvantages of which are described in detail below without employing cost extensive methods like slugging or direct compression.

### BRIEF DESCRIPTION OF DRAWINGS

The foregoing and other features and advantages of the present invention will be better understood from the following detailed description of illustrative embodiments, taken in conjunction with the accompanying drawings in which:

Figure I represents dissolution profiles of tablets in accordance to one aspect of present invention vis-à-vis the marketed formulation, in pH 6.8 phosphate buffer;

Figure II represents dissolution profiles of dose weight proportionate tablets in accordance to one aspect of present invention of 500 mg and 750 mg and 500 mg vis-à-vis 750 mg non-dose weight proportionate tablets of the marketed formulation, in pH 6.8 phosphate buffer;

Figure III represents dissolution profiles of tablets in accordance to one aspect of present invention having single rate controlling polymer vis-a-vis the marketed formulation containing combination of polymers, in pH 6.8 phosphate buffer; and

Figure IV is a schematic representation of the manufacturing process according to the invention.

### DETAILED DESCRIPTION

Compressibility within drug manufacturing is a problem, which not only affects the processing characteristics and machinability of a drug blend but also affects the final compressed tablet form. When a drug, such as metformin, which is poorly compressible and hygroscopic, is manufactured special precautions have to be taken to compress such drugs such as maintaining the relative humidity value below 50% RH during manufacturing and packaging.

It has been surprisingly found that granulating metformin essentially with a binder prior to incorporating in a controlled release matrix increases the compressibility considerably over compositions made using the teachings of prior art. The corresponding increase of hardness values and decrease of friability demonstrated these benefits. More surprising it has been found that the addition of the same concentration of binder to metformin blend including extended release materials and granulating it together, again resulted in poorly compressible granules thereby highlighting the need for essentially granulating metformin with a suitable binder.

Metformin being bitter in nature may be film coated for taste masking and it is observed that the tablets of the present invention, due to their low friability are suitable for film coating and are also suitable for large-scale packing operations.

In the present invention metformin is pre-granulated with a binder and it is only mixed with the rate controlling polymers and a small amount of free metformin. As the polymers are not wetted and remain in dry state, their performance is maximized and hence they can be employed in lower quantities and yet obtain dissolution profiles matching the marketed formulation which may require higher quantities of extended release materials.

Tablets formulated according to the invention have been found to have a tablet weight reduction of at least 20% over marketed formulation. This is an important advantage in improving patient compliance. Moreover due to the reduced weight it is also possible to make dose weight proportionate tablets for the varying strengths of metformin tablets such as 500 mg, 750 mg, 1000 mg and so on. Those skilled in the art in commercial pharmaceutical production will appreciate the advantages obtained by having dose weight proportionate formulations since such formulations dramatically reduce the operation costs and labor involved in manufacturing separate dose dependent blends.

Compositions of the present invention comprising metformin granules essentially bound with about 0.1% to about 10% binder, preferably about 0.2% to about 5% binder and most preferably about 0.25% to about 4.5% binder and dispersed in a rate-controlling matrix of hydrophilic polymers and free metformin drug are explained hereunder in greater detail with respect to individual components and their working ranges.

Although biguanides such as phenformin or buformin or pharmaceutically acceptable salts thereof, may be used for the purpose of the this invention, the preferred drug, having high water solubility for use herein is metformin or pharmaceutically acceptable salts such as metformin hydrochloride, metformin fumarate, and metformin succinate. Metformin can be used in varying doses such as 500 mg, 750 mg, 850 mg, 1000 mg. It is contemplated within the scope of the invention that other pharmaceutical compounds having like characteristics can be formulated into extended release dosages by using the compositional techniques of the present invention. It is also contemplated within the scope of the invention that where desired, metformin or a salt thereof may be used in combination with another antihyperglycemic agent, which may be administered orally in the same dosage form in accordance with the present invention.

The present composition according to the invention is essentially comprised of one or more binders in an amount within the range of from about 0.1% to about 10% binder, preferably about 0.2% to about 5% binder and most preferably about 0.25% to about 4.5% binder by weight of the composition. Binders usually are low viscosity polymers or non-polymeric materials and therefore they do not extend the release of a drug. Although binders improve appearance, hardness & friability of the preparation they are usually not intended to influence the disintegration or dissolution roles of active substance.

Binders which are suitable for use herein include but are not limited to copovidone which is manufactured by free-radical polymerization of 6 parts of vinylpyrrolidone and 4 parts of vinyl acetate in isopropanol. Copovidone is a white or yellowish-white spray-dried powder that has a relatively fine particle size and good flow properties. It has a typical slight odour and a faint taste in aqueous solutions. Because of the ratio of vinylpyrrolidone to vinyl acetate in copovidone, it is almost as universally soluble as polyvinyl pyrrolidone. It dissolves in extremely hydrophilic liquids such as water as well as in more hydrophobic solvents such as butanol. Copovidone has a molecular weight ranging from about 45000 to about 70000 and is available commercially in different grades and trade names such as Kollidon VA 64.

It is contemplated within the scope of the invention that other binders such as polyvinyl pyrrolidone (PVP) with a molecular weight ranging from about 4000 to about 1500000 and preferably about 30,000-1500000 can be used as a binder. Polyvinylpyrrolidone is available in different grades based on K-value and molecular weights such as polyvinyl pyrrolidone with K value of 24-26, 29-32 or 85-95. Preferably polyvinyl pyrrolidone with K value 85-95 (Plasdone K-90/D®, Kollidon 90F®) can be used in the present invention having high molecular weight (1,000,000-1,500,000) and greater binding capacity.

It is also contemplated that other binders can be used such as but not limited to hydroxypropyl methyl cellulose, hydroxypropyl cellulose, hydroxy ethyl cellulose, polyvinyl alcohol, sodium carboxy methyl cellulose, starches such as corn starch, modified corn starch, sugars, gum acacia and the like.

Metformin hydrochloride granules prepared with copovidone (copolyvidone, Kollidon VA 64) have very good compressibility as demonstrated by examples. Metformin is essentially granulated with suitable binders, the binder concentration ranging from about 0.1% to about 10% binder, preferably about 0.2% to about 5% binder and most preferably about 0.25% to about 4.5% binder. Although concentrations above 4% can give also give binding effect, there is no substantial increase in binding hence higher concentrations are not employed or necessary.

The solvent used with the binder for granulation is preferably water. It is contemplated that other solvents such as isopropyl alcohol or the like can also be employed. Metformin granules so formed are uniformly dispersed in a controlled release matrix comprising of rate controlling polymers along with free metformin.

“Controlled-release” as used herein to describe a method and composition for making an active ingredient available to the biological system of a host. A controlled release preparation according to the present invention is one that achieves slow release of a drug over an extended period of time, thereby extending the duration of drug action over that achieved by conventional delivery. This admixture is typically compressed under pressure to produce a tablet. Drug is released from this tablet by diffusion and erosion. For drugs of relatively high solubility, the preferred polymeric matrices are those with a relatively high molecular weight. With such systems, release of the drug is achieved by allowing the gastric fluid to diffuse into the matrix where fluid dissolves the matrix-held drug and then diffuses outward while the matrix retains its integrity, or disintegrates at a rate that is considerably slower than the rate at which the drug is dissolved from matrix. Controlled release is thus achieved by the integrity of the matrix and the need for the gastric fluid to diffuse into the matrix to reach the drug.

In the present invention swelling and expanding system is employed. The controlled release gastro-retentive swelling system of the present invention employs a combination of rate controlling polymers, which swell voluminously in presence of gastric contents to increase the dosage form size such that it precludes its passage through the pylorus. The term “rate-controlling polymer” as used herein includes hydrophilic polymers that are capable of retarding the release of metformin hydrochloride in vivo when metformin hydrochloride is dispersed in a polymeric matrix formed from the rate controlling polymers.

Preferred polymers for the controlled release system of high solubility drug of the present invention are those which ensure rapid hydration of the polymer matrix to minimize variable and significant burst of drug, yet effectively control the release of drug being liberated from the discrete particles or drug granules. The hydrophilic water-soluble polymers may be used individually or in combination. Examples of polymers suitable for this invention include the polymers well known in the pharmaceutical art for their release retarding properties and may be selected from the group comprising acrylic polymers such as available as Eudragit RS, Eudragit RL, natural gums as xanthan gum, karaya gum, locust bean gum, guar gum, gelan gum, gum arabic, tragacanth, carrageenan, pectin, carboxymethyl cellulose (CMC) agar, alginic acid, sodium alginate polyvinylpyrrolidine, hydroxypropylcellulose, hydroxypropylmethyl cellulose, methyl cellulose, vinyl acetate copolymers, polyethylene oxide, methacrylic acid copolymers, maleic anhydride/methyl vinyl ether copolymers and derivatives and mixtures thereof.

Preferred polymers with appropriate hydration characteristics include hydroxypropylmethylcellulose 2208 USP (hydroxypropylmethylcellulose with a methoxyl content of 19-24% and a hydroxypropyl content of 7-12%), viscosity grades ranging from about 4000 to about 100,000 cps and hydroxypropylmethylcellulose 2910 USP (hydroxypropylmethylcellulose with a methoxyl content of 28-30% and a hydroxypropyl content of 7-12%), viscosity grades ranging from about 3 to about 150 cps. Another preferred polymer is sodium carboxy methylcellulose having viscosity of about 2000-50000 cps.

The amount of polymer relative to the drug may vary depending on the release rate desired, nature of the polymers and their physicochemical characteristics. The amount of the polymer in the dosage form generally varies from about 10% to about 50% by weight of the composition. Preferably, the amount of polymers varies from about 15% to about 45% by weight of the dosage form. The polymer concentration can be reduced as they are utilized optimally due to their incorporation in dry form.

Additional excipients that are although not essential for the present invention, are required for the tableting process as known to those skilled in art, and may be suitably included.

The composition of the invention therefore typically includes pharmaceutically acceptable excipients. As is well known to those skilled in the art, pharmaceutical excipients are routinely incorporated into solid dosage forms. This is done to ease the manufacturing process as well as to improve the performance of the dosage form. Common excipients include diluents, lubricants, granulating aids, colorants, flavorants, surfactants, pH adjusters, anti-adherents and gildants etc. Such excipients are routinely used in the dosage forms of this invention.

The present invention may additionally include one or more fillers or excipients in an amount within the range of from about 0 to about 90% by weight and preferably from about 1 to about 80% by weight such as lactose, sugar, corn starch, modified corn starch, mannitol, sorbitol, inorganic salts such as calcium carbonate and/or cellulose derivatives such as wood cellulose and microcrystalline cellulose.

As the composition is in the form of a tablet, it may include one or more tableting lubricants in an amount within the range of from about 0.2 to about 8% and preferably from about 0.5 to about 2% by weight of the composition, such as magnesium stearate, stearic acid, palmitic acid, calcium stearate, talc, polyethylene glycol, colloidal silicon dioxide, sodium stearyl fumarate, carnauba wax and the like and mixtures thereof. Other conventional pharmaceutical ingredients, which may optionally be present, include preservatives, stabilizers, and FD &C colors etc.

The composition made according to the present invention may be formulated as tablets within a capsule or a tablet. Most preferably, the composition is a tablet. The tablet may optionally be coated with a thin layer of a film forming polymer or a pharmaceutical excipient. The controlled release preparation according to the invention may conveniently be film coated using any film coating material conventional in the pharmaceutical art. Preferably an aqueous film coating is used.

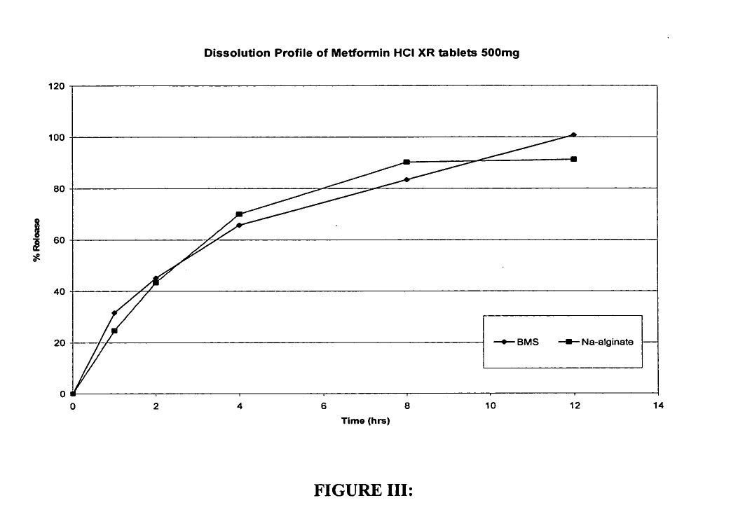

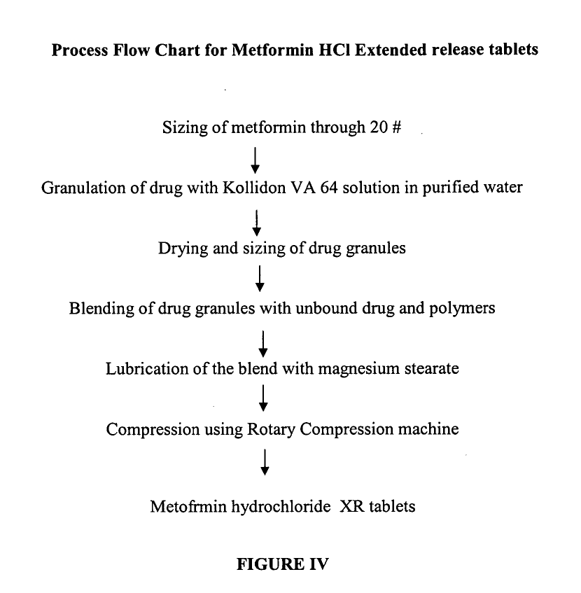

The controlled release, gastro retentive system of present invention can be prepared in accordance with the following method of the invention as shown in FIG. 4. A mixture essentially of metformin hydrochloride and a suitable binder such as copovidone is granulated with a suitable solvent such as water to produce substantially uniform granules. The granules are then dried and passed through a 1.5 to 2 mm aperture screen to break down agglomerates. The resulting dry drug granules are blended with one or more hydrophilic polymers and approximately 2 percent of the total dosage of metformin in free form. The resulting mix may optionally be mixed with diluents or fillers and finally may be lubricated with lubricant before pressing into tablets.

The dosage form of present invention is a solid dosage form, preferably a tablet, which may vary in shape such as oval, triangle, almond, peanut, parallelogram, pentagonal, hexagonal, trapezoidal. The preferred shapes are oval and parallelogram forms.

A controlled release preparation according to the present invention is one that achieves slow release of a drug over an extended period of time, thereby extending the duration of drug action over that achieved by conventional delivery. Preferably, such a preparation maintains a drug concentration in the blood within the therapeutic range for 12 hours or more.

Tablets formulated according to the invention allow for controlled release metformin hydrochloride over at least a twelve-hour period following oral administration, the in vitro release rate preferably corresponds to the following % rate of metformin hydrochloride released as shown in Table 1:

TABLE 1

| TIME (H) | % RELEASED |
|---|---|
| 1 | 10-40 |
| 2 | 20-75 |
| 4 | 30-85 |
| 6 | 50-90 |
| 8 | 60-95 |
| 12 | 65-100 |

Yet another preferred preparation particularly suited for once-a-day dosing has an in-vitro release rate corresponding to the following % rate of metformin hydrochloride released as shown in Table 2:

TABLE 2

| TIME (H) | % RELEASED |
|---|---|
| 1 | 00-50 |
| 2 | 30-75 |
| 4 | 40-85 |
| 6 | 50-100 |
| 8 | 60-100 |
| 12 | 65-100 |

A still father preferred preparation in accordance with the invention is also particularly suited for once-a-day dosing has an in vitro release rate corresponding to the following % rate of metformin hydrochloride released as shown in Table 3:

TABLE 3

| TIME (H) | % RELEASED |
|---|---|
| 1 | 0-30 |
| 2 | 0-40 |
| 4 | 5-55 |
| 6 | 10-65 |
| 8 | 20-75 |
| 12 | 30-90 |
| 16 | 50-100 |
| 24 | >80 |

Another preferred dissolution rate in vitro upon release of the controlled release preparation for administration twice daily according to the invention is between 5 and 50% (by weight) metformin hydrochloride released after 1 hour, between 10 and 75% (by weight) metformin hydrochloride released after 2 hours, between 20 and 95% (by weight) metformin hydrochloride released after 4 hours, between 40 and 100% (by weight) metformin hydrochloride released after 8 hours, more than 50% (by weight) metformin hydrochloride released after 12 hours, more than 70% (by weight) released after 18 hours and more than 80% (by weight) metformin hydrochloride released after 24 hours.

A formulation in accordance with the invention suitable for once a day or twice a day dosing and may have a Tₘₐₓ of 3 to 10 hours, preferably 2 to 7 hours.

While the present invention has been described in terms of its specific embodiments, certain modifications and equivalents will be apparent to those skilled in the art and are intended to be included within the scope of the present invention. The details of the invention, its objects and advantages are explained hereunder in greater detail in relation to non-limiting exemplary illustrations.

### EXAMPLE I

Demonstrates Essential Requirement of Metformin HCl to be Granulated Alone with Binder:

The following composition was prepared according to the process of the present invention where metformin HCl was granulated alone with a binder copovidone and in another experiment a conventional wet granulation process of the prior art is followed wherein all the ingredients were mixed together before granulation.

TABLE 1

| Composition | % w/w | |
|---|---|---|
| Metformin HCl | 50.0 | |
| Copovidone (Kollidon VA 64) | 0.5 | |
| Sodium carboxy methyl cellulose (Cekol 30,000) | 10.0 | |
| Hydroxy propyl methyl cellulose (Methocel K100M) | 29.0 | |
| Hydroxy propyl methyl cellulose (Methocel E5 LV premium) | 7.0 | |
| Microcrystalline cellulose (Avicel PH 10s2) | 3.0 | |
| Magnesium stearate | 0.5 | |
| Demineralized Water* | q.s. | |
| Tablet weight | 1000 | mg |

*Not present in final product

Process: 1: Process According to the Invention

Copovidone was dissolved in de-mineralized water forming a solution and metformin HCl was granulated using this solution. Granules thus obtained were dried in fluidized bed dryer at about 45° C. for about 15 min to achieve LOD of 1-4%. These granules were then sieved through 16# and blended with remaining excipients. The blend was lubricated and compressed into tablets.

Process 2: Granulation of all Excipients Together

Metformin HCl was dry mixed with all excipients except magnesium stearate and granulated using solution of copovidone. Granules thus obtained were dried in fluidized bed dryer at 45° C. for 15 min to achieve LOD of 1-4%. These granules were then sieved through 16#, lubricated and compressed into tablets. Table 2 shows the properties of the granules and tablets obtained with these processes:

TABLE 2

| Property | Process 1 | Process 2 |
|---|---|---|
| Bulk density (g/ml) | 0.5 | 0.52 |
| Tapped density (g/ml) | 0.68 | 0.57 |
| % compressibility | 26.47 | 8.77 |
| Hardness (Kg/cm²) | 11.5-13 | 3.5-7 |
| Friability (100 rev) | 0.12 | 0.94 |
| Friability (500 rev) | 0.53 | 1.82 |

The data suggests that the tablets obtained by process of the invention had superior compressibility, higher hardness value and lower friability values.

### EXAMPLE II

Demonstrates Essential Requirement of a Binder for Metformin HCl Granulation:

The following composition was prepared according to the process of the present invention where metformin HCl was granulated alone with the binder copovidone and in another trial metformin was granulated with water and subsequently mixed with other excipients.

TABLE 3

| | % w/w | | | |
|---|---|---|---|---|
| Composition | A | | B | |
| Metformin HCl | 50.0 | | 50.0 | |
| Copovidone (Kollidon VA 64) | 0.5 | | — | |
| Sodium carboxy methyl cellulose (Cekol 30,000) | 10.0 | | 10.0 | |
| Hydroxy propyl methyl cellulose (Methocel | 29.0 | | 29.0 | |
| K100M) | | | | |
| Hydroxy propyl methyl cellulose (Methocel E5 | 7.0 | | 7.0 | |
| LV premium) | | | | |
| Microcrystalline cellulose (Avicel PH 102) | 3.0 | | 3.0 | |
| Magnesium stearate | 0.5 | | 0.5 | |
| Demineralized Water* | q.s. | | q.s. | |
| Tablet weight | 1000 | mg | 1000 | mg |

*Not present in final product

Process According to the Invention

For formulation A copovidone was dissolved in de-mineralized water forming a solution and metformin HCl was granulated using this solution. In formulation B metformin HCl was granulated with water without use of a binder. Granules thus obtained in both formulations were dried in fluidized bed dryer at 45° C. for 15 min to achieve LOD of 1-4%. These granules were then sieved through 16# and mixed with remaining excipients. The blend was lubricated and compressed into tablets. The following table shows the properties of the granules and tablets obtained with these processes:

TABLE 4

| Property | Formulation A | Formulation B |
|---|---|---|
| Bulk density (g/ml) | 0.5 | 0.56 |
| Tapped density (g/ml) | 0.68 | 0.75 |
| % compressibility | 26.47 | 25.33 |
| Hardness (Kg/cm²) | 11.5-13.0 | 6.5-7.5 |
| Friability (100 rev) | 0.12 | 0.26 |
| Friability (500 rev) | 0.53 | 2.62 |

The data suggests that the tablets obtained by granulation of metformin HCl without a binder had poor compressibility, lower hardness value and higher friability values. This clearly demonstrates that metformin HCl is required to be granulated separately with a binder for tablets having improved properties.

### EXAMPLE III

Demonstrates Essential Requirement of a Wet Granulation Using Binder for Metformin HCl Granulation:

The following composition was prepared according to the process of the present invention where Metformin HCl is granulated alone with the binder, copovidone and a conventional direct compression process of prior art where all the ingredients were mixed together before compression.

TABLE 5

| | % w/w | | | |
|---|---|---|---|---|
| Composition | A | | B | |
| Metformin HCl | 50.0 | | 50.0 | |
| Copovidone (Kollidon VA 64) | 0.25 | | 0.25 | |
| Sodium carboxy methyl cellulose (Cekol 30,000) | 10.0 | | 10.0 | |
| Hydroxy propyl methyl cellulose (Methocel | 29.0 | | 29.0 | |
| K100M) | | | | |
| Hydroxy propyl methyl cellulose (Methocel E5 | 7.0 | | 7.0 | |
| LV premium) | | | | |
| Microcrystalline cellulose (Avicel PH 102) | 3.3 | | 3.3 | |
| Magnesium stearate | 0.5 | | 0.5 | |
| Demineralized Water* | q.s. | | q.s. | |
| Tablet weight | 1000 | mg | 1000 | mg |

*Not present in final product

Process According to the Invention

For formulation A copovidone was dissolved in de-mineralized water forming a solution and metformin HCl was granulated using this solution. Granules thus obtained were dried in fluidized bed dryer at 45° C. for 15 min to achieve LOD of 1-4%. These granules were then sieved through 16# and mixed with remaining excipients. The blend was lubricated and compressed into tablets. Whereas in formulation B metformin HCl was dry mixed with all excipients and used blend directly for compression. The following table shows the properties of the granules and tablets obtained with these processes:

TABLE 6

| Property | Formulation A | Formulation B |
|---|---|---|
| Bulk density (g/ml) | 0.52 | 0.54 |
| Tapped density (g/ml) | 0.68 | 0.81 |
| % compressibility | 23.53 | 33.33 |
| Hardness (Kg/cm²) | 11.0-13.0 | 5.5-7.0 |
| Friability (100 rev) | 0.00 | 0.17 |
| Friability (500 rev) | 0.33 | 3.75 |

The data suggests that the tablets obtained by direct compression of metformin HCl with all excipients had poor compressibility, lower hardness value and higher friability values. This demonstrates that metformin HCl is required to be wet granulated separately with a binder for tablets having improved properties.

### EXAMPLE IV

Demonstrates the Superior Effect of Compositions of Present Invention in Terms of Compressibility Over Prior Art Compositions:

The following compositions were made in accordance with the methods listed in the prior art and compared with the compositions of the present invention in terms of % compressibility, friability (100 and 500 revolutions) and hardness.
Composition A: Present invention
Composition B: All excipients granulated together with water
Composition C: All excipients granulated with PVP K30 as per US 20030104059A1
Composition D: Direct compression as per U.S. Pat. No. 6,524,618
Composition E: Slugging as per US2004005900A1
Composition F: Granulation using wax
Composition G: As per formula given in BMS patent

The compositions A-G are tabulated in Table 6 as follows:

TABLE 7

| Ingredients | A | B | C | D | E | F | G |
|---|---|---|---|---|---|---|---|
| Metformin | 50.0 | 50.0 | 68.8 | 50 | 50 | 50 | 50 |
| hydrochloride | | | | | | | |
| Copovidone | 0.5 | 0.5 | — | — | — | — | — |
| (Kollidone VA 64) | | | | | | | |
| Polyvinyl pyrrolidone | — | — | 1.6 | — | — | — | — |
| (Kollidone K30) | | | | | | | |
| Cetyl alcohol | — | — | — | — | — | 2.5 | — |
| Sodium CMC | 10.0 | 10.0 | 4.0 | — | 3.6 | 10.0 | 5.0 |
| (Cekol 30000) | | | | | | | |
| HPMC | 29.0 | 29.0 | 12.0 | — | 36.5 | 29.0 | 38.5 |
| (Methocel K100M) | | | | | | | |
| HPMC | 7.0 | 7.0 | — | — | — | 7.0 | 1 |
| (Methocel E5) | | | | | | | |
| Polyox WSR 303 | — | — | — | 30 | — | — | — |
| Microcrystalline | — | — | 13.3 | — | 6.0 | — | — |
| Cellulose PH101 | | | | | | | |
| Microcrystalline | 1.0 | 1.0 | | — | — | 1.0 | 10.2 |
| Cellulose PH102 | | | | | | | |
| Lactose DCL15 | — | — | — | 19.0 | — | — | — |
| Mag. Stearate | 0.5 | 0.5 | 1.2 | 0.5 | 0.6 | 0.5 | 1.1 |
| Aerosil | — | — | — | 0.5 | | — | — |

Process for Composition A

Copovidone was dissolved in de-mineralized water forming a solution and metformin HCl was granulated using this solution. Granules thus obtained were dried in fluidized bed dryer at 45° C. for 15 min to achieve LOD of 1-4%. These granules were then sieved through 16# and mixed with remaining excipients. The blend was lubricated and compressed into tablets.

Process for Composition B

Metformin HCl was dry mixed with all excipients except magnesium stearate and granulated using solution of copovidone. Granules thus obtained were dried in fluidized bed dryer at 45° C. for 15 min to achieve LOD of 1-4%. These granules were then sieved through 16#, lubricated and compressed into tablets.

Process for Composition C

Metformin HCl and all other excipients were mixed together and granulated using a solution of PVP K30 in water. Granules thus obtained were dried in fluidized bed dryer at 45° C. for 15 min to achieve LOD of 1-4%. These granules were then sieved through 16 #, lubricated and compressed into tablets.

Process for Composition D

Metformin HCl and all excipients were sieved and blended together. The blend was then lubricated and compressed into tablets.

Process for Composition E

Metformin HCl hydrochloride and MCC were mixed together in a blender. The mass was then hydrated with about 3% of water. The mass thus obtained was further mixed with other excipients and lubricated and compressed into 16 mm tablets that were further broken down to give granules which were lubricated and compressed into final tablets.

Process for Composition F

Cetyl alcohol was melted and metformin HCl was granulated using molten cetyl alcohol. These granules were then sieved through 16# and mixed with remaining excipients. The blend was lubricated and compressed into tablets.

Process for Composition G

Metformin HCl hydrochloride was dry mixed with Cekol 30000 and granulated using de-mineralized water. Granules thus obtained were dried in fluidized bed dryer at 45° C. for 15 min to achieve LOD of 1-4%. These granules were then sieved through 16# and mixed with remaining excipients. The blend was lubricated and compressed into tablets.

The characteristics of the lubricated granules obtained by different processes as well as tablet characteristics are listed below in Table 8:

TABLE 8

| Parameters | A | B | C | D | E | F | G |
|---|---|---|---|---|---|---|---|
| Bulk density | 0.5 | 0.52 | 0.54 | 0.66 | 0.50 | 0.57 | 0.55 |
| (g/ml) | | | | | | | |
| Tapped | 0.68 | 0.57 | 0.64 | 0.79 | 0.68 | 0.69 | 0.74 |
| density | | | | | | | |
| (g/ml) | | | | | | | |
| % compres- | 26.47 | 8.77 | 15.6 | 16.4 | 26.4 | 26.09 | 26.02 |
| sibility | | | | | | | |
| Hardness | 11.5-13 | 3.5-7 | 4-5 | 6 | 8.5-10 | 4-4.5 | 6.5-7 |
| (Kg/cm²) | | | | | | | |
| Friability | 0.12 | 0.94 | 0.99 | 0.20 | 1.0 | 0.85 | 0.48 |
| (100 rev) | | | | | | | |
| Friability | 0.53 | 1.82 | Failed | 1.17 | Failed | Failed | 3.43 |
| (500 rev) | | | Tablets | | Tablets | Tablets | |
| | | | broken | | broken | broken | |

$\% Compressibility = \frac{( Tapped Bulk density - Poured bulk density )}{Tapped bulk density} \times 100$

It is evident from the data within table 8 that compositions of the present invention, as represented by composition A, have good % compressibility of the blend and superior friability and hardness on compression over prior art compositions. The results of friability after 500 revolutions demonstrate the feasibility of the compositions of the present invention to be coated for taste masking.

### EXAMPLE V

Demonstrates Minimum Quantity of Binder to be Employed for Metformin Hcl Granulation:

The following composition was prepared according to the process of the present invention

TABLE 10

| Composition | % w/w | |
|---|---|---|
| Metformin HCl | 50.0 | |
| Copovidone (Kollidon VA 64) | 0.25 | |
| Sodium carboxy methyl cellulose (Cekol 30,000) | 10.0 | |
| Hydroxy propyl methyl cellulose (Methocel K100M) | 29.0 | |
| Hydroxy propyl methyl cellulose (Methocel E5 LV premium) | 7.0 | |
| Microcrystalline cellulose (Avicel PH 102) | 3.2 | |
| Magnesium stearate | 0.5 | |
| Demineralized Water* | q.s. | |
| Tablet weight | 1000 | mg |

*Not present in final product

Process According to the Invention

Copovidone was dissolved in de-mineralized water forming a solution and metformin HCl was granulated using this solution. Granules thus obtained were dried in fluidized bed dryer at 45° C. for 15 min to achieve LOD of 1-4%. These granules were then sieved through 16# and mixed with remaining excipients. The blend was lubricated and compressed into tablets.

The following table shows the properties of the granules and tablets obtained with this process:

TABLE 11

| Property | Formulation |
|---|---|
| Bulk density (g/ml) | 0.52 |
| Tapped density (g/ml) | 0.68 |
| % compressibility | 23.53 |
| Hardness (Kg/cm²) | 11.0-13.0 |
| Friability (100 rev) | 0.01 |
| Friability (500 rev) | 0.33 |

The data suggests that tablets obtained by granulation of metformin HCl with low percentage of a binder had good compressibility, high hardness value and lower friability values. This example demonstrates that copovidone acts as a binder even at percentage of 0.25% with respect to the tablet weight.

### EXAMPLE VI

Demonstrates that Different Binders Employed in the Selected Range Does Not Act as Release Rate Retarding Polymer:

The binders used for this example include copovidone (Kollidon VA 64), polyvinylpyrrolidone (Kollidon 90F), and Sodium CMC (Cekol 30000). The general formula employed for these compositions is listed in Table 12:

TABLE 12

| Composition | % used | mg/Tablet |
|---|---|---|
| Metformin HCl | 98 | 500 |
| Binder | 1 | 5 |
| Demineralized water | qs | qs |
| Magnesium stearate | 1 | 5 |

General Process Employed:

Metformin HCl was granulated with aqueous solution of the binder. Granules thus obtained were dried in fluidized bed dryer at 45° C. for 15 min to achieve LOD of 1-4%. These granules were then sieved through 16#, lubricated and compressed into tablets.

In vitro dissolution of these tablets was carried out using USP dissolution apparatus I (Basket) at 100 rpm with 900 ml phosphate buffer pH 6.8 as medium. The data so obtained is tabulated in Table 13:

TABLE 13

| | % dissolved | % dissolved |
|---|---|---|
| Binder | after 15 min | after 30 min |
| Copovidone (Kollidon VA 64) | 100.40 | 99.90 |
| Polyvinylpyrrolidone 90F | 100.83 | 101.62 |
| (Kollidon K90F) | | |
| Sodium CMC (Cekol 30000) | 98.88 | 99.22 |

The above data suggests that the binders employed for granulation of metformin HCl, does not retard the release of metformin HCl. It also indicates that the initial burst of metformin HCl can be easily curbed by plain metformin HCl-with-binder granulations dispersed in controlled release matrix and disadvantageous granulations with rate controlling polymers need not be employed.

### EXAMPLE VII

Demonstrate that Copovidone Does Not Act as Release Rate Retarding Polymer at Different Concentrations:

Copovidone was used as binder at different concentrations 1%, 5%, 10%, 30%, 50% and 150% w/w of metformin. The formulae employed for these compositions are listed in Table 14:

TABLE 14

| | % w/w | | | | | |
|---|---|---|---|---|---|---|
| Composition | A | B | C | D | E | F |
| Metformin HCl | 98 | 94.3 | 90 | 76 | 66 | 39.5 |
| Binder | 1 | 4.7 | 9 | 23 | 33 | 59.5 |
| Demineralized water | qs | qs | qs | qs | qs | qs |
| Magnesium stearate | 1 | 1 | 1 | 1 | 1 | 1 |

General Process Employed:

Metformin HCl was granulated with aqueous solution of the binder at 1% w/v concentration. For other formulations, the remaining amount of copovidone was added in the dry blend along with other excipients. Granules thus obtained were dried in fluidized bed dryer at 45° C. for 15 min to achieve LOD of 1-4%. These granules were then sieved through 16#, lubricated and compressed into tablets.

In vitro dissolution of these tablets was carried out using USP dissolution apparatus I (Basket) at 100 rpm with 900 ml phosphate buffer pH 6.8 as medium. The data so obtained is tabulated in Table 15:

TABLE 15

| | % dissolved | % dissolved |
|---|---|---|
| Binder | 15 min | 30 min |
| Formulation A | 100.4 | 99.9 |
| Formulation B | 100.2 | 102.1 |
| Formulation C | 95.7 | 97.0 |
| Formulation D | 97.1 | 97.9 |
| Formulation E | 96.7 | 106.1 |
| Formulation F | 55.4 | 92.3 |

The above data indicates that the binder employed for granulation of metformin HCl, does not retard the release of metformin HCl even at a high concentration of 60% w/w (Tablet weight 1250 mg). Concentrations above this are not practicable as it increases the weight of the tablet to a size which is difficult to swallow.

### EXAMPLE VIII

Demonstrates Optimized Use of Polymers Resulting in Dose Weight Proportionate Tablets:

Metformin blended according to the process of the present invention is prepared using the following composition as seen in Table 16:

TABLE 16

| Composition | % w/w |
|---|---|
| Metformin HCl | 68.49 |
| Polyvinyl pyrrolidone (Kollidon K90F) | 3.65 |
| Sodium carboxy methyl cellulose | 9.14 |
| (Cekol 30,000) | |
| Hydroxy propyl methyl cellulose K100M | 18.26 |
| (Methocel K100M) | |
| Magnesium stearate | 0.46 |
| Demineralized Water* | q.s. |

*Not present in final product

Metformin is dry mixed with polyvinyl pyrrolidone and granulated using de-mineralized water. Granules thus obtained are dried in fluidized bed dryer at 45° C. for 15 min to achieve LOD of 1-4%. These granules are then sieved through 20# and mixed with remaining excipients. The blend is lubricated and compressed into tablets. The same granules are compressed at 730 mg weight for 500 mg and 1095 mgs for 750 mg strengths.

Dissolution studies in pH 6.8 phosphate buffer using USP dissolution apparatus I are carried out for the 500 mg and 750 mg metformin tablets of the present invention and Glucophage® XR of both strengths.
Medium: 900 ml pH 6.8 phosphate buffer
Dissolution apparatus: USP apparatus-Type I

The results are as listed in Table 17:

TABLE 17

| | Glucophage | | Glucophage | |
|---|---|---|---|---|
| | XR 500 mg | | XR 750 mg | MET |
| Time (hrs) | (MBM57) | MET XR 500 mg | (Rx-766201) | XR 750 mg |
| 0 | 0 | 0 | 0 | 0 |
| 1 | 31.68 | 32.80 | 30.51 | 29.31 |
| 2 | 45.17 | 51.25 | 44.81 | 44.55 |
| 4 | 65.76 | 72.48 | 64.10 | 65.06 |
| 6 | 70.24 | 85.52 | 78.64 | 78.82 |
| 8 | 83.40 | 91.17 | 86.97 | 87.43 |
| 12 | 100.73 | 95.07 | 93.36 | 90.53 |
| | | F2 = 54 | | F2 = 88.9 |

Weight of 500 mg Glucophage XR tablet is 1000 mg and that of Glucophage XR 750 mg is 1095 mg indicating that they are not dose weight proportional probably due to higher amount of polymers employed for retarding release of 500 mg/750 mg tablets. However, as demonstrated in the previous example, it is possible to reduce the weight of the tablet in the present invention and therefore develop a dose weight formulation for 500 mg and 750 mg strengths.

### EXAMPLE IX

Demonstrates Rate Controlling Effect of Single Polymer Comparable to Marketed Formulation:

A composition is made using the following ingredients as listed in Table 14 and prepared using the process of the present invention as shown in example I. Sodium alginate is used singly as rate controlling agent as opposed to the multiple polymers used by the marketed formulation.

TABLE 18

| Composition | % w/w |
|---|---|
| Metformin HCl | 55.6 |
| Polyvinyl pyrrolidone K90F (Kollidon K90F) | 2.8 |
| Sodium alginate (Keltrone HVCR) | 27.8 |
| Microcrystalline cellulose (Avicel 102) | 13.3 |
| Magnesium stearate | 0.6 |
| Demineralized Water* | q.s. |

*Not present in final product

Dissolution studies in pH 6.8 phosphate buffer using USP dissolution apparatus I show following release profile as seen in Table 19:

TABLE 19

| Time (hrs) | % released | Marketed formulation |
|---|---|---|
| 0 | 0 | 0 |
| 1 | 24.71 | 31.68 |
| 2 | 43.41 | 45.17 |
| 4 | 70.06 | 65.76 |
| 8 | 90.30 | 83.40 |
| 12 | 91.27 | 100.0 |
| F2 value | 60 | |

It appears in Figure III, that optimum usage of polymers in the composition of the present invention enables polymers to be used alone for controlling the release and yet match the profiles of the marketed formulation.

### EXAMPLE X

Demonstrates Metformin Particle Size Independent Compressibility of Present Compositions:

Compositions A, B and C are made using different particle sizes of Metformin as listed below:

| | |
|---|---|
| Composition A: | Present invention (crystalline drug fines 10%) |
| Composition B: | Fine powdered drug (Fines - 50%) |
| Composition C: | Fine powdered drug (Fines - 100%) |

Fines = Amount of drug passing through 80# sieve

Composition made using the following ingredients as listed in Table 20 is prepared using the process of the present invention as mentioned hereunder.

TABLE 20

| S. no | Ingredients | % w/w |
|---|---|---|
| 1 | Metformin hydrochloride | 50.0 |
| 2 | Polyvinyl pyrrolidone (Kollidon 90F) | 2.5 |
| 3 | Sodium CMC (Cekol 30000) | 10.0 |
| 4 | HPMC (Methocel K100M) | 29.0 |
| 5 | HPMC (Methocel E5 LV) | 7.0 |
| 6 | Microcrystalline Cellulose (Avicel PH102) | 1.0 |
| 7 | Magnesium Stearate | 0.5 |
| | Tablet weight | 1000 mg |

Process for Compositions A, B, C

Metformin is dry mixed with polyvinyl pyrrolidone (Kollidon K90F) and granulated using de-mineralized water. Granules thus obtained are dried in fluidized bed dryer at 45° C. for 15 min to achieve LOD of 1-4%. These granules are then sieved through 16# and mixed with remaining excipients. The blend is lubricated and compressed into tablets. Except particle size of Metformin HCl (with respect to fines) used for granulation all excipients were same in composition. The characteristics of the granules obtained by different processes as well as tablet charateristics are listed below in Table 21:

TABLE 21

| Parameters | A | B | C |
|---|---|---|---|
| Bulk density (g/ml) | 0.43 | 0.51 | 0.52 |
| Tapped density (g/ml) | 0.59 | 0.72 | 0.68 |
| % Compressibility | 26.7 | 29.17 | 23.53 |
| Hardness (Kg/cm²) | 12 | 10 | 11 |
| Friability (100 rev) | 0.019 | 0.19 | 0.21 |
| Friability (500 rev) | 0.27 | 0.88 | 0.93 |

It appears that granulation of metformin with a binder significantly improves the processability of the API and the process becomes independent of the physical characteristics of the API like particle size, which is very crucial for prior art processes such as direct compression.

### EXAMPLE XI

In-Vivo Study

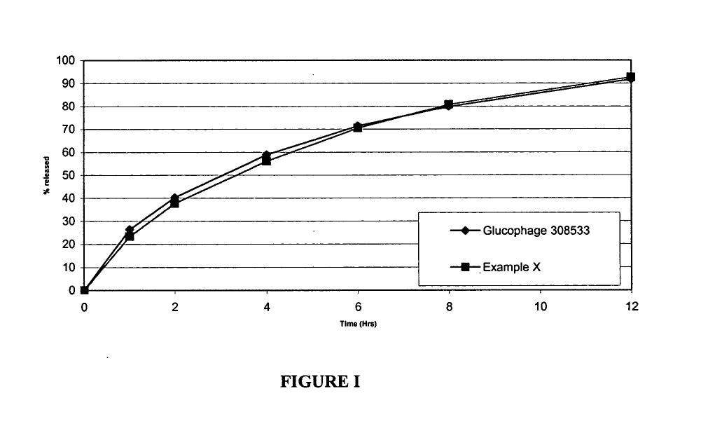

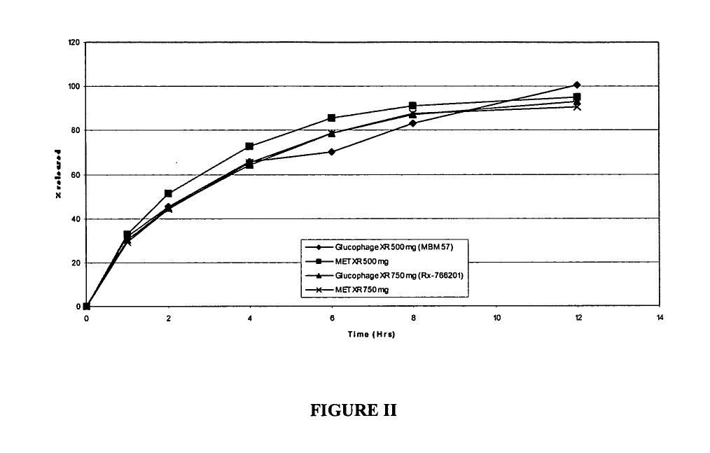

Tablets containing 500 mg metformin hydrochloride are prepared according to composition of example X (In vitro release profile in FIG. 1) using process A and Glucophage XR tablets 500 mg is dosed (1×500 mg tablets) to eight patients immediately after high fat breakfast. Blood samples are collected at 0, 1.0, 2.0, 3.0, 3.5, 4.0, 4.5, 5.0, 5.5, 6.0, 6.5, 7.0, 7.5, 8.0, 8.5, 9.0, 12.0, 14.0, 24.0 and 36.0 hours and analyzed for metformin hydrochloride. The mean plasma profile demonstrated no impact on bioavailability as compared to innovator-Glucophage XR® 500 mg. Inter-patient variability in pharmacokinetic parameters was acceptable as illustrated by the mean parameters (% CV) given in the table below:

TABLE 22

| Formulation | Cmax (ng/ml) | AUC (inf) (ng · hr/ml) | Tmax (hr) |
|---|---|---|---|
| Glucophage XR | 696.579 ± 101.8837 | 8377.837 ± 1563.8892 | 6.000 ± 1.4832 |
| 500 mg | | | |
| Example X | 756.389 ± 69.8909 | 8228.283 ± 1659.1882 | 6.167 ± 1.4024 |

### EXAMPLE XII

Formulation of Metformin According to the Invention.

I. Metformin HCl XR Tablets 500 mg

Manufacturing Formula For Metformin HCl XR Tablets 500 mg

TABLE 23

| | | |
|---|---|---|
| Sr. no | Ingredients | % w/w |

Intragranular addition

| | | |
|---|---|---|
| 1 | Metformin hydrochloride | 49.0 |
| 2 | Copovidone (Kollidon VA 64) | 0.25 |
| 3 | DM water* | q.s. |

Extragranular addition

| | | |
|---|---|---|
| 4 | Metformin HCl | 1.00 |
| 5 | Copovidone (Kollidon VA 64) | 0.25 |
| 6 | Sodium CMC (Cekol 30000) | 3.80 |
| 7 | HPMC (Methocel K100M) | 19.5 |
| 8 | Microcrystalline Cellulose (Avicel PH102) | 25.7 |
| 9 | Magnesium Stearate | 0.5 |
| | Tablet weight for 500 mg strenth | 1000 mg |

*not present in the final product

TABLE 24
Physical parameters of the tablet

| | |
|---|---|
| Hardness (kg/cm²) | 10-12.5 |
| Friability (%) | |
| 100 rev | 0.12 |
| 500 rev | 0.59 |

II. Metformin HCl XR Tablets 750 mg

Manufacturing Formula for Metformin HCl XR Tablets 750 mg

TABLE 25

| | | | |
|---|---|---|---|
| Sr. no | Ingredients | mg/tab | % w/w |

Intragranular addition

| | | | |
|---|---|---|---|
| 1 | Metformin hydrochloride | 735.00 | 66.82 |
| 2 | Copovidone (Kollidon VA 64) | 3.75 | 0.34 |
| 3 | DM water* | q.s | q.s. |

Extragranular addition

| | | | |
|---|---|---|---|
| 4 | Metformin HCl | 15.00 | 1.34 |
| 5 | Copovidone (Kollidon VA 64) | 4.25 | 0.39 |
| 6 | Sodium CMC (Cekol 30000) | 63.00 | 5.73 |
| 7 | HPMC (Methocel K100M) | 190.00 | 17.27 |
| 8 | Microcrystalline Cellulose (Avicel PH102) | 83.00 | 7.55 |
| 9 | Magnesium Stearate | 5.00 | 0.45 |
| 10 | Iron oxide yellow | 1.00 | 0.09 |
| | Tablet weight for 500 mg strength | 1100.00 | 100 |

*not present in the final product

TABLE 26
Physical parameters of the tablet

| | |
|---|---|
| Hardness (kg/cm²) | 10.5-12.0 |
| Friability (%) | |
| 100 rev | 0.26 |
| 500 rev | 1.39 |

Steps are same as for 500 mg strength tablet except extra granular blending step where iron oxide is added.

### EXAMPLE XIII

Metformin Sustained Release Formulation using Combination of Methocel and Polyox

TABLE 27

| Composition | % w/w |
|---|---|
| Metformin HCl | 62.50 |
| Copovidone (Kollidon VA 64) | 0.33 |
| Sodium carboxy methyl cellulose (Cekol 30,000) | 1.25 |
| Hydroxy propyl methyl cellulose (Methocel K100M) | 18.33 |
| Polyoxyethylene WSR 303 | 4.83 |
| Microcrystalline cellulose (Avicel PH 102) | 12.33 |
| Magnesium stearate | 0.42 |
| Demineralized Water* | q.s. |

*Not present in final product

Process According to the Invention

In this formulation, copovidone was dissolved in de-mineralized water forming a solution and metformin HCl was granulated using this solution. Granules thus obtained were dried in fluidized bed dryer at 45° C. for 15 min to achieve LOD of 1-4%. These granules were then sieved through 16# and mixed with remaining excipients. The blend was lubricated and compressed into tablets.

It will be understood that various modifications may be made to the embodiments and examples disclosed herein. Therefore, the above description and examples should not be construed as limiting, but merely as exemplification of the various embodiments. Those skilled in the art will envision other modifications within the scope and spirit of the claims appended hereto.

What is claimed:

1. An oral dosage form of metformin or a pharmaceutically acceptable salt thereof wherein the hardness of said oral dosage form is at least about 8 kg/cm².

2. The dosage form of claim 1 wherein metformin or pharmaceutically acceptable salt thereof is in the form of granules.

3. The dosage form of claim 1 wherein metformin or pharmaceutically acceptable salt thereof further comprises:
about 0.1 to about 10% binder; and
a rate-controlling matrix of hydrophilic polymers wherein said metformin or pharmaceutically acceptable salt thereof is substantially bound with said binder forming granules, said granules are further dispersed in the rate-controlling matrix of hydrophilic polymers.

4. The oral dosage form of claim 3 wherein said binder is selected from the group consisting of copovidone, polyvinyl pyrrolidone, hydroxy propyl methyl cellulose, hydroxy propyl cellulose, hydroxy ethyl cellulose, polyvinyl alcohol and sodium carboxy methyl cellulose.

5. The oral dosage form of claim 3 wherein said binder copovidone.

6. The oral dosage form of claim 5 further comprising one or more tableting lubricants in an amount within the range of from about 0.2 to about 8%

7. The oral dosage form of claim 5 further comprising from about 0.1 to about 4% by weight of the total dosage form of metformin or pharmaceutically acceptable salt thereof not bound to said binder.

8. The oral dosage form of claim 7 wherein said unbound metformin or pharmaceutically acceptable salt thereof comprises about 2% by weight.

9. The oral dosage form of claim 1 wherein said hydrophilic polymers is selected from the group consisting of Eudragit RS, Eudragit RL, xanthan gum, karaya gum, locust bean gum, guar gum, gelan gum, gum arabic, tragacanth, carrageenan, pectin, carboxymethyl cellulose, agar, alginic acid, sodium alginate polyvinylpyrrolidine, hydroxypropylcellulose, hydroxypropylmethyl cellulose, methyl cellulose, vinyl acetate copolymers, polyethylene oxide, methacrylic acid copolymers, maleic anhydride/methyl vinyl ether copolymers and derivatives and mixtures thereof.

10. The oral dosage form of claim 1 wherein said hydrophilic polymers is selected from the group consisting of hydroxypropylmethylcellulose 2208 USP, hydroxypropylmethylcellulose 2910 USP, sodium carboxy methylcellulose and mixtures thereof.

11. A process for preparing an oral dosage form of metformin or a pharmaceutically acceptable salt thereof wherein the hardness of said oral dosage form is at least about 8 kg/cm² comprising steps of
(i) granulating metformin with 0.1 to about 10% binder
(ii) dispersing the resulting granules in one or more rate-controlling hydrophilic polymers; and
(iii) compressing the composition so obtained into tablets of at least about 8 kg/cm².

12. The process of claim 11 further comprising the step of adding about 0.5 % to about 4% of unbound metformin or pharmaceutically acceptable salt thereof wherein said unbound metformin is dispersed within said one or more rate-controlling hydrophilic polymers.

13. The process of claim 11 further comprising the step of adding about 2% of unbound metformin or pharmaceutically acceptable salt thereof wherein said unbound metformin is dispersed within said one or more rate-controlling hydrophilic polymers.

14. The process of claim 11 wherein said binder is selected from the group consisting of copovidone, polyvinyl pyrrolidone, hydroxy propyl methyl cellulose, hydroxy propyl cellulose, hydroxy ethyl cellulose, polyvinyl alcohol and sodium carboxy methyl cellulose.

15. The process of claim 11 wherein said binder copovidone.

16. The process of claim 11 further comprising the step of adding one or more tableting lubricants in an amount within the range of from about 0.2 to about 8%.

17. The process of claim 11 said hydrophilic polymers is selected from the group consisting of Eudragit RS, Eudragit RL, xanthan gum, karaya gum, locust bean gum, guar gum, gelan gum, gum arabic, tragacanth, carrageenan, pectin, carboxymethyl cellulose, agar, alginic acid, sodium alginate polyvinylpyrrolidine, hydroxypropylcellulose, hydroxypropylmethyl cellulose, methyl cellulose, vinyl acetate copolymers, polyethylene oxide, methacrylic acid copolymers, maleic anhydride/methyl vinyl ether copolymers and derivatives and mixtures thereof.

18. The process of claim 11 said hydrophilic polymers is selected from the group consisting of hydroxypropylmethylcellulose 2208 USP, hydroxypropylmethylcellulose 2910 USP, sodium carboxy methylcellulose and mixtures thereof.
